(19) United States
(12) Patent Application Publication
(10) Pub. No.: US 2022/0268748 A1
(43) Pub. Date: Aug. 25, 2022

(54) PORTABLE SYSTEM FOR MEASURING AND ANALYZING INDOOR AIR QUALITY AND METHOD THEREOF

(71) Applicant: Manasa Hari Bhimaraju, Cupertino, CA (US)

(72) Inventor: Manasa Hara Bhimaraju, Cupertino, CA (US)

(21) Appl. No.: 17/182,875

(22) Filed: Feb. 23, 2021

(51) Int. Cl. G01N 33/00; H04L 29/08; G16Y 20/10; G08B 5/22; G06F 3/14

(52) U.S. Cl. CPC G01N 33/0075; G01N 33/0063; H04L 67/12; G16Y 20/10; G08B 5/22; G06F 3/14; G01N 2033/0068; H04L 67/02

(57) ABSTRACT

The present invention discloses a portable system for measuring and analyzing indoor air quality and method thereof The system comprises an Internet Of Things (IOT) enabled sensor device (101) for collecting a user's geospatial data, wherein the sensor device (101) comprises a plurality of sensors for collecting indoor air quality data at pre-defined intervals of time. Further, a data analysis and storage module (102) is employed for receiving the indoor air quality data collected by the sensor device (101), wherein the data analysis and storage module (102) automatically analyses the data which is subsequently stored on a remote server (102a) for future requirements. Additionally, a User Interface (UI) module (103) is employed for displaying the analyzed data obtained from the remote server (102a) through a web-based application installed on a user interface device.

### DESCRIPTION OF THE INVENTION

### Technical Field of the Invention

The present invention discloses a portable system for measuring and analyzing indoor air quality and method thereof. The invention particularly relates to a portable sensor device for measuring a plurality of pollutants and conditions in an indoor environment, wherein the measured indoor air quality is analyzed and stored on a cloud-based infrastructure and displayed through a User Interface (UI) module for user's reference.

### Background of the Invention

Several agencies have monitored, collected, and stored outdoor air quality data. However, the indoor air quality, which is quite different from outdoor air quality, has been largely overlooked. Common indoor air pollutants include Particulate Matter (PM) 2.5, PM 10, Carbon Dioxide (CO₂), Nitrogen Dioxide (NO₂), Carbon Monoxide (CO), Volatile Organic Compounds (VOCs), Ozone (O₃), Liquid Petroleum Gas (LPG), Natural Gas (NG), Formaldehyde (HCHO), and biological contaminants like bacteria/fungi. A set of existing solutions monitoring the indoor air quality have been surveyed but these solutions fail to explain how to interpret the data, how it affects the user's health, and remedies on how to improve their indoor air quality. Additionally, the existing systems store the air quality data and user data for a limited time period thereby limiting the scope of further research pertaining to the analyzed and stored data. Further, these solutions are expensive, lack data accessibility features, and cover only a limited set of indoor air pollutants.

The Patent Application No. US20160116181A1 titled “Indoor air quality sense and control system” discloses an indoor air quality (IAQ) system for sensing and controlling air quality within a structure is provided. The IAQ system includes a plurality of air quality sensor modules configured to sense IAQ parameters and remotely located within the structure. The IAQ system also includes an IAQ control hub including (i) a communication interface communicatively coupling the IAQ control hub to the plurality of air quality sensor modules and (ii) memory holding instructions that cause a processor to receive the IAQ parameters from the plurality of air quality sensor modules and if one of the IAQ parameters is outside a predetermined IAQ parameter range corresponding to the one of the IAQ parameters, request adjustment of control settings of a heating, ventilation, and air conditioning (HVAC) system to shift indoor air quality toward the predetermined IAQ parameter range. However, the invention does not disclose details pertaining to the portability of the IAQ system.

The Patent No. US20150052975A1 titled “Networked air quality monitoring” discloses systems, methods, and non-transitory computer-readable media for continuously monitoring residential air quality and providing a trend based analysis regarding various air pollutants are presented herein. The system comprises an air quality monitor located in a residential house, wherein the air quality monitor is configured to measure the level of an air pollutant. The system also includes a server that is communicatively coupled to the air quality monitor, wherein the server is configured to generate a unique environmental fingerprint associated with the residential house. However, the invention does not provide suggestions for improving indoor air quality which is highly essential for ensuring that the indoor pollutant levels are maintained within a pre-defined threshold.

Hence, there exists a need for a portable system for measuring and analyzing indoor air quality.

### SUMMARY OF THE INVENTION

The present invention overcomes the drawbacks of the prior art by disclosing a portable system for measuring and analyzing indoor air quality and method thereof. The system comprises an Internet Of Things (TOT) enabled sensor device for collecting a user's geospatial data, wherein the sensor device comprises a plurality of sensors for collecting indoor air quality data at pre-defined intervals of time. Further, a data analysis and storage module is employed for receiving the indoor air quality data collected by the sensor device, wherein the data analysis and storage module automatically analyses the data, which is subsequently stored on a remote server for future requirements. Additionally, a User Interface (UI) module is employed for displaying the analyzed data obtained from the remote server through a web-based application installed on a user interface device.

The present invention provides a portable sensor device for measuring indoor air quality data, wherein the sensor device is capable of measuring a plurality of pollutants and conditions such Particulate Matter (PM) 2.5, PM10, CO₂, NO₂, CO, VOCs, O₃, Liquid Petroleum Gas (LPG), Natural Gas (NG), Equivalent Carbon Dioxide (eCO₂), Hydrogen, Temperature, Humidity, Pressure, Altitude and so on. Further, the present invention provides a data analysis and storage module, which is a cloud-based infrastructure that stores and analyzes the indoor air quality and relatively provides suggestions for improving the indoor air quality, which reflects on a healthy lifestyle. Additionally, the system is a useful tool for the public health domain as it provides the qualitative and quantitative data, which allows users to manage their indoor air quality in a cost-effective and hassle-free manner. Furthermore, the present invention provides a secure interface for retrieving the indoor air quality data without revealing any user related information without the user's consent. This feature provides several new avenues of research in public health in drawing connections between air quality data and lifestyle and helps discover any possible patterns between air quality data and other health conditions. This is a nascent research area requiring a lot of indoor air quality data which is currently not available in other existing solutions.

The methods, systems, and devices are set forth in part in the description which follows, and in part will be obvious from the description, or can be learned by practice of the methods, apparatuses, and systems. The advantages of the methods, devices, and systems will be realized and attained by means of the elements and combinations particularly pointed out in the appended claims. It is to be understood that both the foregoing general description and the following detailed description are exemplary and explanatory only and are not restrictive of the methods, devices, and systems, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

The foregoing and other features of embodiments will become more apparent from the following detailed description of embodiments when read in conjunction with the accompanying drawings. In the drawings, like reference numerals refer to like elements.

FIG. 1 illustrates a block diagram of a system for measuring and analyzing indoor air quality.

FIG. 2 illustrates a method for analyzing indoor air quality.

FIG. 3 illustrates block diagram for a method for sending alerts to users regarding the indoor air quality.

### DETAILED DESCRIPTION OF THE INVENTION

Reference will now be made in detail to the description of the present subject matter, one or more examples of which are shown in figures. Each example is provided to explain the subject matter and not a limitation. Various changes and modifications obvious to one skilled in the art to which the invention pertains are deemed to be within the spirit, scope and contemplation of the invention.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The present invention discloses a portable system for measuring and analyzing indoor air quality and method thereof The system comprises an Internet Of Things (TOT) enabled sensor device for collecting a user's geospatial data, wherein the sensor device comprises a plurality of sensors for collecting indoor air quality data at pre-defined intervals of time. Further, a data analysis and storage module is employed for receiving the indoor air quality data collected by the sensor device, wherein the data analysis and storage module automatically analyses the data which is subsequently stored on a remote server for future requirements. Additionally, a User Interface (UI) module is employed for displaying the analyzed data obtained from the remote server through a web-based application installed on a user interface device.

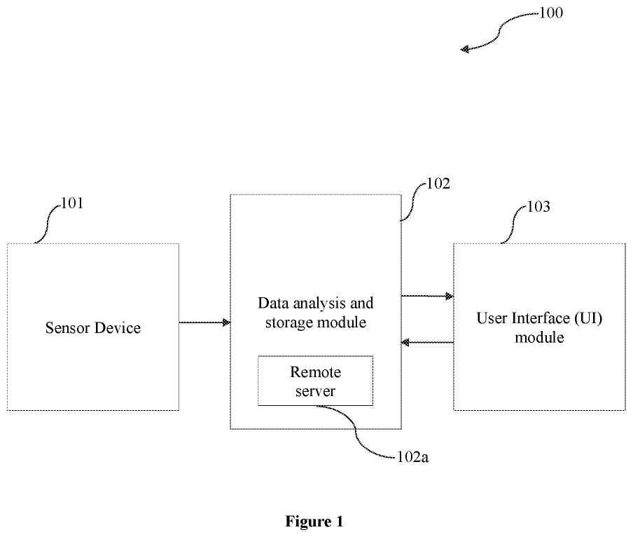

FIG. 1 illustrates a block diagram of a system for measuring and analyzing indoor air quality, wherein the system (100) comprises an Internet Of Things (TOT) enabled sensor device (101) for collecting a user's geospatial data which includes pollutants and conditions such as

Particulate Matter (PM) 2.5, PM10, CO₂, NO₂, CO, VOCs, O₃, Liquid Petroleum Gas (LPG), Natural Gas (NG), Equivalent Carbon Dioxide (eCO₂), Hydrogen, temperature, humidity, pressure, altitude and so on. The sensor device (101) comprises a plurality of sensors for collecting indoor air quality data at pre-defined intervals of time, wherein the sensors present in the sensor device (101) are calibrated using linear regression and machine learning algorithms to obtain high accuracy of the sensors in the sensor device (101). The sensor device (101) starts collecting indoor air quality data upon initiation of the sensor device (101) through the web-based application on a user interface device such as a laptop, desktop, tablet, smartphone and so on. In one embodiment, the sensor device (101) may be used for monitoring outdoor air quality in addition to monitoring indoor air quality. The sensor device (101) may comprise at least one microcontroller which is programmed to allow the plurality of sensors in the sensor device (101) to collect indoor air quality data.

In one embodiment, the sensor device (101) may be a standard model which measures indoor pollutants such as PM2.5, PM10, CO, O₃, CO₂, eCO₂, total volatile organic compounds (tVOCs), any traces of LPG, methane, temperature, pressure, altitude, humidity and so on, wherein the standard model of the sensor device (101) may be priced at a pre-defined low price point. In another embodiment, the sensor device (101) may be a comprehensive model which has the capability of measuring indoor pollutant and condition such as hydrogen, flammable gases, aromatic compounds, hydrogen sulfide, ammonia, nitrogen oxide, natural gas, formaldehyde and so on in addition to the pollutants and conditions measured by the standard model of the sensor device (101), wherein the comprehensive model of the sensor device (101) may be priced at a higher price point compared to the standard model of the sensor device (101).

Further, the collected geospatial data is encrypted by the sensor device (101) and transmitted to a data analysis and storage module (102) through a wired or wireless network infrastructure such as Local Area Network (LAN), Wi-Fi and so on wherein the data analysis and storage module (102) is a cloud-based infrastructure which receives the indoor air quality data collected by the sensor device (101) and automatically analyses the data which is subsequently stored on a remote server (102a) for future requirements. In one embodiment, the data analysis and storage module (102) analyzes and stores the analyzed air quality data on the remote server (102a) for a pre-defined long period time which enables future research possibilities using the stored data. The encrypted geospatial data from the sensor device (101) is locally stored on the sensor device (101) during the absence of a wired or wireless network infrastructure and is automatically deleted upon transmission to the remote server (102a) upon detecting the presence of a wired or wireless network infrastructure. This feature is particularly useful as a public health research tool as the sensor device (101) may be used to collect indoor air quality data for low-income groups that cannot afford an internet connection and store the data locally till the presence of a wired or wireless network infrastructure is detected.

Further, the analyzed indoor air quality data stored on the remote server (102a) in the data analysis and storage module (102) is decrypted and transmitted to a User Interface (UI) module (103) for displaying the analyzed data obtained from the remote server (102a) through the web-based application installed on a user interface device. In one embodiment, the web-based application which interfaces with the sensor device (101) may employ Restful Application Programming Interface (REST API) for interacting with the data analysis and storage module (102), wherein the REST API uses Hypertext Transfer Protocol (HTTP) requests to interact with the remote server (102a). The web-based application which interfaces with the sensor device (101) may send the measured and encrypted indoor air quality data to the data analysis and storage module (102) using a HTTP POST request, wherein the data analysis and storage module (102) analyzes the indoor air quality data and stores the analyzed data on the remote server (102a). Subsequently, the UI module (103) may send an HTTP GET request to the data analysis and storage module (102) which decrypts the analyzed indoor air quality data and transmits the data to the UI module (103) for display. However, the present invention may use alternate APIs for interfacing between different modules of the system (100).

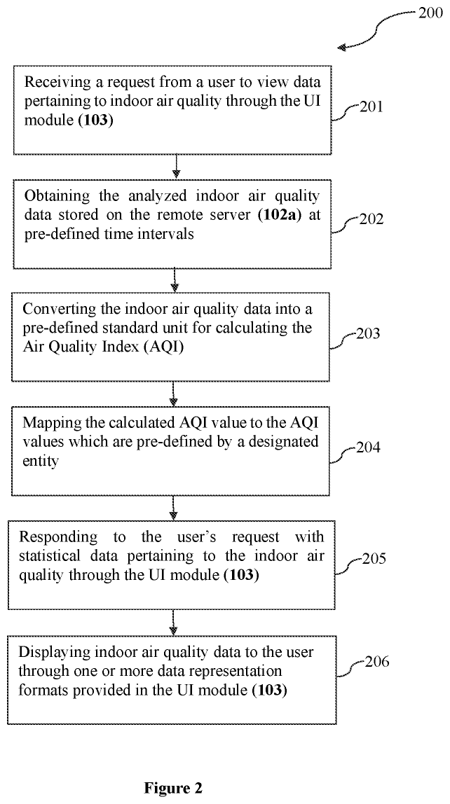

FIG. 2 illustrates a method for analyzing indoor air quality, wherein the method (200) comprises the steps of receiving a request from a user to view data pertaining to indoor air quality through the UI module (103) in step (201). In one embodiment, such as request may be a HTTP GET request sent by the UI module (103) to the data analysis and storage module (102). In step (202), the analyzed indoor air quality data stored on the remote server (102a) is obtained at pre-defined time intervals subsequent to which the obtained indoor air quality data is converted into a pre-defined standard unit for calculating the Air Quality Index (AQI) such as ug/m3 and so on in step (203). In step (204), the calculated AQI value is mapped with the AQI values which are pre-defined by a designated entity such as a government entity. The resultant statistical data pertaining to the indoor air quality is displayed to the user through the UI module (103) in step (205). Subsequently, indoor air quality data is displayed to the user through one or more data representation formats provided in the UI module (103) in step (206).

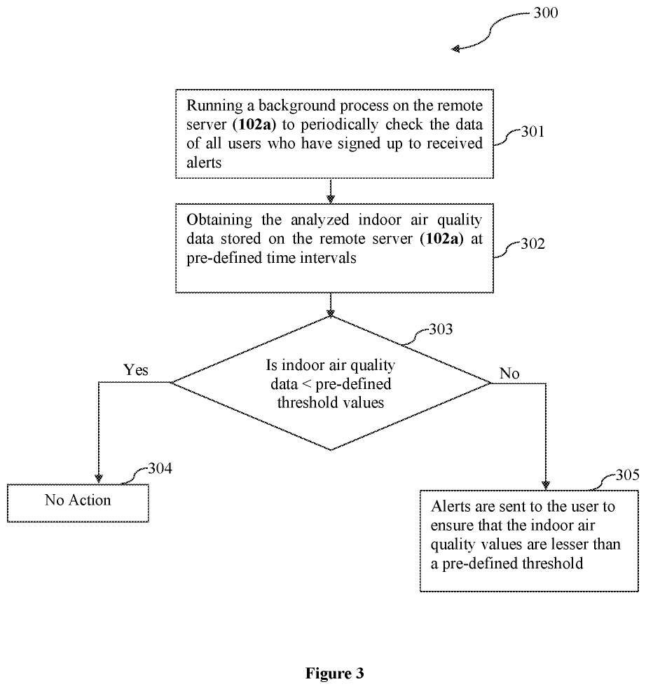

FIG. 3 illustrates a method for sending alerts to users regarding the indoor air quality, wherein the method (300) comprises the steps of running a background process on the remote server (102a) to periodically check the data of all users who have signed up to received alerts in step (301). Subsequently, in step (302), the indoor air quality data stored on the remote server (102a) is obtained at pre-defined time intervals. In step (303), the analyzed indoor air quality data is compared with a pre-defined threshold value. Resultantly, if the analyzed air quality data is lesser than a pre-defined threshold value, no alerts are sent to the users in step (304).

Conversely, if the analyzed air quality data is greater than a pre-defined threshold value, one or more alerts such as e-mails, texts and so on are sent to the user for allowing the user to take appropriate measures to ensure that the indoor air quality values are lesser than a pre-defined threshold in step (305). In one embodiment, the UI module (103) may provide an hourly dashboard which indicates the level of pollutants and other conditions for the user's reference. A gauge may reflect the calculated indoor AQI index as per the conventions of the user's country. If the statistical data pertaining to the indoor air quality is not satisfactory, the UI module (103) provides suggestions to the user for controlling the indoor pollution levels thereby improving the user's quality of life. Additionally, the UI module (103) allows the users to download the indoor air quality data for future reference or investigation.

The present invention provides a portable sensor device (101) for measuring indoor air quality data, wherein the sensor device (101) is capable of measuring a plurality of pollutants and conditions such Particulate Matter (PM) 2.5, PM10, CO₂, NO₂, CO, VOCs, O₃, Liquid Petroleum Gas (LPG), Natural Gas (NG), Equivalent Carbon Dioxide (eCO₂), Hydrogen, Temperature, Humidity, Pressure, Altitude and so on. Further, the present invention provides a data analysis and storage module (102) which is a cloud-based infrastructure that stores and analyzes the indoor air quality and relatively provides suggestions for improving the indoor air quality which reflects on a healthy lifestyle. Additionally, the system (100) is a useful tool for the public health domain as it provides the qualitative and quantitative data which allows users to manage their indoor air quality in a cost-effective and hassle-free manner. Furthermore, the present invention provides a secure interface for retrieving the indoor air quality data without revealing any user related information without the user's consent. This feature provides several new avenues of research in public health in drawing connections between air quality data and lifestyle and helps discover any possible patterns between air quality data and other health conditions. This is a nascent research area requiring a lot of indoor air quality data which is currently not available in other existing solutions.

While at least one exemplary embodiment has been presented in the foregoing detailed description, it should be appreciated that a vast number of variations exist.

As used in this application, the term “system” is intended to refer to a computer-related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

Generally, program modules include routines, programs, components, data structures, etc., that perform particular tasks or implement particular abstract data types. Moreover, those skilled in the art will appreciate that the inventive methods can be practiced with other computer system configurations, including single-processor or multiprocessor computer systems, minicomputers, mainframe computers, as well as personal computers, hand-held computing devices, microprocessor-based or programmable consumer electronics, and the like, each of which can be operatively coupled to one or more associated devices.

The illustrated aspects of the innovation may also be practiced in distributed computing environments where certain tasks are performed by remote processing devices that are linked through a communications network. In a distributed computing environment, program modules can be located in both local and remote memory storage devices.

A computer typically includes a variety of computer-readable media. Computer-readable media can be any available media that can be accessed by the computer and includes both volatile and nonvolatile media, removable and non-removable media. By way of example, and not limitation, computer-readable media can comprise computer storage media and communication media. Computer storage media includes volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or any other medium which can be used to store the desired information and which can be accessed by the computer.

Communication media typically embodies computer-readable instructions, data structures, program modules or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

Software includes applications and algorithms. Software may be implemented in a smart phone, tablet, or personal computer, in the cloud, on a wearable device, or other computing or processing device. Software may include logs, journals, tables, games, recordings, communications, SMS messages, Web sites, charts, interactive tools, social networks, VOIP (Voice Over Internet Protocol), e-mails, and videos.

In some embodiments, some or all of the functions or process(es) described herein and performed by a computer program that is formed from computer readable program code and that is embodied in a computer readable medium. The phrase “computer readable program code” includes any type of computer code, including source code, object code, executable code, firmware, software, etc. The phrase “computer readable medium” includes any type of medium capable of being accessed by a computer, such as read only memory (ROM), random access memory (RAM), a hard disk drive, a compact disc (CD), a digital video disc (DVD), or any other type of memory.

### Reference Numbers:

| Components | Reference Numbers |
|---|---|
| System | 100 |
| Sensor device | 101 |
| Data analysis and storage module | 102 |
| Remote server | 102a |
| User Interface (UI) module | 103 |

I claim:

1. A portable system for measuring and analyzing indoor air quality, the system (100) comprising:
a. an Internet Of Things (TOT) enabled sensor device (101) for collecting a user's geospatial data, wherein the sensor device (101) comprises a plurality of sensors for collecting indoor air quality data at pre-defined intervals of time;
b. a data analysis and storage module (102) which receives the indoor air quality data collected by the sensor device (101) and automatically analyses the data which is subsequently stored on a remote server (102a) for future requirements;
c. a User Interface (UI) module (103) for displaying the analyzed data obtained from the remote server (102a) through a web-based application installed on a user interface device.

2. The system (100) as claimed in claim 1, wherein the sensor device (101) starts collecting indoor air quality data upon initiation of the sensor device (101) through the web-based application on a user interface device.

3. The system (100) as claimed in claim 1, wherein the sensors present in the sensor device (101) are calibrated using linear regression and machine learning algorithms to obtain high accuracy of the sensors in the sensor device (101).

4. The system (100) as claimed in claim 1, wherein the geospatial data from the sensor device (101) is transmitted to the data analysis and storage module (102) through a wired or wireless network infrastructure.

5. The system (100) as claimed in claim 1, wherein the geospatial data from the sensor device (101) is locally stored on the sensor device (101) during the absence of a wired or wireless network infrastructure.

6. The system (100) as claimed in claim 1, wherein the geospatial data which is locally stored on the sensor device (101) during the absence of a wired or wireless network infrastructure is automatically deleted upon transmission to the remote server (102a) using a wired or wireless network infrastructure.

7. The system (100) as claimed in claim 1, wherein the geospatial data is encrypted by the sensor device (101) and transmitted to the data analysis and storage module (102) for analysis and subsequently stored on the remote server (102a).

8. The system (100) as claimed in claim 1, wherein the encrypted and analyzed data which is stored on the remote server (102a) is decrypted and transmitted to the UI module (103) for the user to view the analyzed geospatial data.

9. A method for analyzing indoor air quality, the method (200) comprising the steps of:
a. receiving a request from a user to view data pertaining to indoor air quality through the UI module (103);
b. obtaining the analyzed indoor air quality data stored on the remote server (102a) at pre-defined time intervals;
c. converting the indoor air quality data into a pre-defined standard unit for calculating the Air Quality Index (AQI);
d. mapping the calculated AQI value to the AQI values which are pre-defined by a designated entity;
e. responding to the user's request with statistical data pertaining to the indoor air quality through the UI module (103);
f. Displaying indoor air quality data to the user through one or more data representation formats provided in the UI module (103).

10. A method for sending alerts to users regarding the indoor air quality, the method (300) comprising the steps of:
a. running a background process on the remote server (102a) to periodically check the data of all users who have signed up to received alerts;
b. obtaining the analyzed indoor air quality data stored on the remote server (102a) at pre-defined time intervals;
c. comparing the analyzed indoor air quality data with a pre-defined threshold value, wherein if the analyzed air quality data is:
i. lesser than a pre-defined threshold value, no alerts are sent to the users;
ii. greater than a pre-defined threshold value, one or more alerts are sent to the user for allowing the user to take appropriate measures to ensure that the indoor air quality values are lesser than a pre-defined threshold.

11. The method (300) as claimed in claim 10, wherein the UI module (103) provides suggestions to the user for controlling the indoor pollution levels thereby improving the user's quality of life.
